(19) United States
(12) Patent Application Publication
(10) Pub. No.: US 2014/0378319 A1
(43) Pub. Date: Dec. 25, 2014

(54) Method To Determine Location, Size and In Situ Conditions In Hydrocarbon Reservoir With Ecology, Geochemistry, and Biomarkers

(71) Applicant: EXXONMOBIL USPSTREAM RESEACH COMPANY, Houston, TX (US)

(72) Inventors: Aaron B. Regberg, Houston, TX (US); A. Lucie N'Guessan, Houston, TX (US); Amelia C. Robinson, Houston, TX (US)

(21) Appl. No.: 14/365,532

(22) PCT Filed: Jan. 11, 2013
(86) PCT No.: PCT/US2013/021258

§ 371 Date: Jun. 13, 2014

Related U.S. Application Data
(60) Provisional application No. 61/595,394, filed on Feb. 6, 2012.

(51) Int. Cl. C12Q 1/64; G01N 33/92; G01N 33/68; C12Q 1/68

(52) U.S. Cl. CPC C12Q 1/64; C12Q 1/6888; G01N 33/92; G01N 33/6842; C12Q 2600/158; C12Q 2600/156; G01N 2570/00
USPC 506/2; 506/9

(57) ABSTRACT

A method of identifying a hydrocarbon system is disclosed A sample from an area of interest is obtained. A first plurality of analyses is used to determine a community structure of an ecology of the sample. A second plurality of analyses is used to determine a community function of the ecology of the sample. The community structure and the community function are used to determine whether the ecology of the sample matches a characteristic ecology of a hydrocarbon system. When the ecology of the sample matches the characteristic ecology, the sample is identified as part of the hydrocarbon system.

### CROSS-REFERENCE TO RELATED APPLICATION

This application claims the benefit of U.S. Provisional Patent Application 61/595,394 filed Feb. 6, 2012 entitled A Method to Determine the Location, Size and In Situ Conditions in a Hydrocarbon Reservoir with Ecology, Geochemistry, and Collections of Biomarkers, the entirety of which is incorporated by reference herein.

### FIELD OF THE INVENTION

The present techniques relate to determining the presence of an active hydrocarbon system, and specifically, to ascertaining the presence, temperature, pressure, salinity and volume of a hydrocarbon reservoir.

### BACKGROUND

This section is intended to introduce various aspects of the art, which may be associated with embodiments of the disclosed techniques. This discussion is believed to assist in providing a framework to facilitate a better understanding of particular aspects of the disclosed techniques. Accordingly, it should be understood that this section is to be read in this light, and not necessarily as admissions of prior art.

The exploration for and discovery of new oil reserves has become increasingly challenging and costly. Untapped reserves tend to be more difficult to identify and evaluate, and are often located subsea, which further increases the complexity and cost of discovering such reserves. Successful, efficient, and cost effective identification and evaluation of hydrocarbon-bearing reservoirs is therefore very desirable.

FIG. 1 depicts a hydrocarbon system, indicated generally at 100. Hydrocarbon system 100 includes an organic carbon bearing source rock 102 that generates and excretes liquid and gaseous hydrocarbons, which migrate through various migration pathways 103 into a reservoir interval 104. The hydrocarbons are trapped in the reservoir interval. A sealing interval above the reservoir interval prevents further hydrocarbon migration out of the reservoir.

In marine exploration, seep detection has become an important tool to identify potential hydrocarbon resources in the subsurface. Oil and gas accumulations often leak hydrocarbons including methane, ethane, propane, butane, naphthalene, and benzene. These hydrocarbons may migrate toward the surface, shown in FIG. 1 as a seafloor 108, through a variety of pathways, such as faults 110 or fracture zones 111. This hydrocarbon migration results in seeps 112 discharging hydrocarbons to the surface. Therefore, the seeps are surface expressions of subsurface geological phenomena. In some instances, seeps may not be directly above the accumulation from which they originate. Seeps may be classified as macro-seeps and micro-seeps, which differ in hydrocarbon flux or areal extent over which the seep discharges.

During hydrocarbon exploration sample sites are chosen by targeting seeps, which are surface expressions of subsurface geological phenomena. Currently discharging seeps (active seeps) or paleo-seeps are typically identified by seismic survey interpretations and may also be located with ship-board sonar. Once a likely site for the hydrocarbon accumulation has been established, an exploration well 114 is drilled. Usually only one core sample 122 is taken at each feature. The core samples are usually several feet in length and are collected below the surface or below the water-sediment interface. The cores are then transported to land-based laboratories for analysis using fluorescence and standard petroleum geochemistry techniques. Because the costs of seep surveys may reach one million U.S. dollars for a forty sample survey, sampling density tends to be quite low. Accordingly, there exists a need for an alternate method to identify currently discharging seeps indicative of active hydrocarbon systems. After drilling, evaluation of the subsurface geology surrounding the well may be achieved through indirect methods such as mud logging and well-based geophysical techniques like electrical conductance, acoustics, and radioactive decay.

While formation evaluation techniques such as well logging remain the standard for the petroleum industry, these techniques are less effective where challenging conditions exist. For instance, there may be cases where the presence of hydrocarbons, fluid type (gas, oil, and/or water), and hydrocarbon water ratio in the pore spaces are ambiguous even after formation evaluation. For example, carbonate reservoirs, thin-bedded clastic rocks, and wells containing very fresh water are particularly troublesome to evaluate using current techniques. Also, current formation evaluation techniques tend to be unable to determine the oil's viscosity, particularly where the oil is biodegraded or severely altered. Contamination or invasion into the formation by hydrocarbon-based drilling fluids is another complication that makes distinguishing the natural hydrocarbon composition and quality using standard logging or geochemical methods much more difficult. Additionally, when wells are drilled and only water is discovered in the potential reservoir unit, the standard formation evaluation techniques do not provide a reliable way to determine whether there are hydrocarbons in an up dip or adjacent position (such as across a fault). Accordingly, there exists a substantial need for reliable, reproducible, efficient, robust, and cost-effective means for identifying and evaluating hydrocarbon-bearing formations. In particular, there exists a substantial need for improving the efficacy and reliability of seep surveys, and to reduce the cost of seep surveys.

FIG. 2 presents a typical workflow 200 for oil and gas exploration and includes pre-drill activities 202 and post-drill activities 204. Pre-drill activities 202, which generally require a lower investment, include selection of a region of interest 206, which may be supported by preliminary seismic information 208 as well as the geologic context 210. Pre-drill activities 202 also include surface feature identification and seabed characterization 212, which involves various types of surface mapping, such as sonar 214, seep detection 216, and drop cores (shallow cores) 218. In contrast to pre-drill activities, post-drill activities 204 are more involved and costly. Subsurface characterization 220 involves drilling exploration wells 222 and the use of 3D seismic 224 where necessary. In addition, a wide array of geochemical analyses of fluids and rocks 226 may be employed. The analyses may be conducted on mud gas 228, drill stem test (DST) 230, refinery samples 232, wireline samples 234, outcrop samples 236, cores 238, cuttings 240, production liquids 242, and seeps 244. In this context, ‘fluids’ refers to pore waters such as those obtained from seafloor sediments from drop cores, liquid hydrocarbons, and formation as well as produced waters. ‘Rocks’ refers to solid material recovered from drilling and includes drill cuttings, conventional cores, sidewall cores and drop cores.

The geochemical analysis 226 is useful for identifying and characterizing the type of oil that is present in a reservoir. The geophysical techniques, such as seismology, electrical resistivity, electro-magnetic techniques and formation evaluation, are used to identify geologic and lithologic structures associated with reservoirs and traps. Occasionally these geophysical techniques even record direct indicators of hydrocarbon reservoirs. However, they often lack the necessary resolution to locate reservoirs or to clearly describe the conditions in a reservoir (pressure, temperature, volume, salinity, hydrocarbon type, etc.). Additionally, geophysical tools provide little information about the type of hydrocarbons present in a reservoir.

The ecology of a hydrocarbon system may provide additional information helpful to oil and gas exploration. Surface features associated with seeps have been linked to mineral precipitation (such as calcium carbonate) as a result of degradation of hydrocarbons at the seawater-sediment interface. Some researchers have coupled morphology of both large and localized mineral precipitate structures (e.g., Beggiatoa mats) with mapping seep, fault and subsurface geology. It is possible to use biological information for exploration and hydrocarbon characterization purposes. Some have produced “lab-on-a-chip” type tools, microarrays or polymerase chain reaction (PCR) methods that, through specific binding of probes, can be indicative of certain target (and previously known) species or function that identifies hydrocarbon degrading capabilities or other catabolic pathways. The premise is that these organisms should be identifiable and more abundant where the greatest volume of hydrocarbons has accumulated.

FIG. 3 depicts an amended workflow 300 for oil and gas exploration showing places within the workflow where ecological analysis of microorganisms may be performed to assess the conditions of a hydrocarbon reservoir. During pre-drill activities 302 water samples (124 in FIG. 1) may be taken at or near a suspected seep 112 to determine the ecology 304 of the associated water column. To control for microorganisms present in the water that are not associated with a seep, a water sample 116 may also be taken in a region where there are no known seeps. Other samples 118 may be taken from shallow sediment on the seafloor to determine the ecology of the seafloor 306. Analysis of samples from the water column and the seafloor yield information about surface feature identification and characterization 308. During post-drilling activities 310, analysis of the subsurface ecology 312 may aid subsurface characterization 314. Samples taken from the reservoir fluids 120 and the drill core 122 help determine the subsurface ecology of fluids and rocks 316, respectively. The ecology of fluids and rocks 316 may also use cuttings 318, production liquids 320, seeps 322, and other core samples 324.

Much of the work used to obtain biological information for hydrocarbon systems has relied on culture-based techniques. These techniques are limited because the vast majority of organisms, particularly those living within a hydrocarbon reservoir, are not able to be cultured. Certainly identifying or finding microbes that have originated in the reservoir and transported to the surface would be ideal, but given the limited number that would possibly survive transport intact, relying on culture-based techniques is not really feasible or representative. In addition, one assumption with earlier studies is that the organisms living in the subsurface are similar to those at the surface. However, recent evidence indicates that the biodiversity in the subsurface is quite complex and many of the subsurface species found have not been identified previously. With increasing genetic divergence from known reference species, PCR and microarrays such as those using oligonucleotide-type probes become less effective. Therefore, many of the probe-based methods may be restricted to finding organisms that have some genetic similarity to known organisms, and therefore potentially miss a large portion of the information obtainable by new methods, such as pyrosequencing and metagenomics.

Application of microbiology-based tracers has been successful where hydrocarbon degradation occurs or is associated with known functions such as bacterial sulfate reduction or reactions that alter fluid properties. By identifying diagnostic organisms or probes associated with a particular function, one can identify whether thermogenic hydrocarbons are present, but information about the pressure, temperature or volume within the reservoir is not really provided. In survey mode, some techniques may identify areal extent of hydrocarbon seepage at the air-sediment interface and then may be used to estimate volume in the subsurface when tied to other tools, such as seismology, to estimate reservoir thickness. One rather significant drawback to this sort of approach, however, is the assumption that migration from a reservoir occurs vertically and in a systematic fashion and that the thermogenic hydrocarbons at the sediment surface are not from anthropogenic contributions, such as a spill or leaky underground storage tank. Any interpretation beyond the presence/absence of an active hydrocarbon system requires some simplification or interpretation of the structural complexity and other geologic phenomena to assess areal extent of the reservoir.

### SUMMARY

In one aspect, a method of identifying a hydrocarbon system is disclosed. A sample from an area of interest is obtained. A first plurality of analyses is used to determine a community structure of an ecology of the sample. A second plurality of analyses is used to determine a community function of the ecology of the sample. The community structure and the community function are used to determine whether the ecology of the sample matches a characteristic ecology of a hydrocarbon system. When the ecology of the sample matches the characteristic ecology, the sample is identified as part of the hydrocarbon system.

### DESCRIPTION OF THE FIGURES

The foregoing and other advantages of the disclosed methodologies and techniques may become apparent upon reviewing the following detailed description and drawings of non-limiting examples of embodiments in which:

FIG. 1 is a cross section view of a hydrocarbon system and an associated seafloor seep;

FIG. 2 is a block diagram of a workflow describing known methods and techniques used in hydrocarbon exploration;

FIG. 3 is a block diagram of the workflow of FIG. 2 updated to use ecological information for hydrocarbon exploration;

FIG. 4 is a schematic diagram of a workflow according to methodologies and techniques described herein;

FIG. 5 is a chart describing in situ and ex situ analyses used to describe the ecology of a sample;

FIG. 6 is a schematic detailing different types of seafloor hydrocarbon seeps;

FIG. 7 is a schematic diagram showing interrelationships of various ecologies.

### DETAILED DESCRIPTION OF THE DISCLOSURE

To the extent the following description is specific to a particular embodiment or a particular use, this is intended to be illustrative only and is not to be construed as limiting the scope of the invention. On the contrary, it is intended to cover all alternatives, modifications, and equivalents that may be included within the spirit and scope of the invention.

Some portions of the detailed description which follows are presented in terms of procedures, steps, logic blocks, processing and other symbolic representations of operations on data bits within a memory in a computing system or a computing device. These descriptions and representations are the means used by those skilled in the data processing arts to most effectively convey the substance of their work to others skilled in the art. In this detailed description, a procedure, step, logic block, process, or the like, is conceived to be a self-consistent sequence of steps or instructions leading to a desired result. The steps are those requiring physical manipulations of physical quantities. Usually, although not necessarily, these quantities take the form of electrical, magnetic, or optical signals capable of being stored, transferred, combined, compared, and otherwise manipulated. It has proven convenient at times, principally for reasons of common usage, to refer to these signals as bits, values, elements, symbols, characters, terms, numbers, or the like.

Unless specifically stated otherwise as apparent from the following discussions, terms such as obtaining, using, determining, identifying, or the like, may refer to the action and processes of a computer system, or other electronic device, that transforms data represented as physical (electronic, magnetic, or optical) quantities within some electrical device's storage into other data similarly represented as physical quantities within the storage, or in transmission or display devices. These and similar terms are to be associated with the appropriate physical quantities and are merely convenient labels applied to these quantities.

Embodiments disclosed herein also relate to an apparatus for performing the operations herein. This apparatus may be specially constructed for the required purposes, or it may comprise a general-purpose computer selectively activated or reconfigured by a computer program or code stored in the computer. Such a computer program or code may be stored or encoded in a computer readable medium or implemented over some type of transmission medium. A computer-readable medium includes any medium or mechanism for storing or transmitting information in a form readable by a machine, such as a computer (‘machine’ and ‘computer’ are used synonymously herein). As a non-limiting example, a computer-readable medium may include a computer-readable storage medium (e.g., read only memory (“ROM”), random access memory (“RAM”), magnetic disk storage media, optical storage media, flash memory devices, etc.). A transmission medium may be twisted wire pairs, coaxial cable, optical fiber, or some other suitable transmission medium, for transmitting signals such as electrical, optical, acoustical or other form of propagated signals (e.g., carrier waves, infrared signals, digital signals, etc.)).

Furthermore, modules, features, attributes, methodologies, and other aspects can be implemented as software, hardware, firmware or any combination thereof. Wherever a component of the invention is implemented as software, the component can be implemented as a standalone program, as part of a larger program, as a plurality of separate programs, as a statically or dynamically linked library, as a kernel loadable module, as a device driver, and/or in every and any other way known now or in the future to those of skill in the art of computer programming. Additionally, the invention is not limited to implementation in any specific operating system or environment.

Example methods may be better appreciated with reference to flow diagrams. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional blocks not shown herein. While the figures illustrate various actions occurring serially, it is to be appreciated that various actions could occur in series, substantially in parallel, and/or at substantially different points in time.

Various terms as used herein are defined below. To the extent a term used in a claim is not defined below, it should be given the broadest possible definition persons in the pertinent art have given that term as reflected in at least one printed publication or issued patent.

As used herein, “and/or” placed between a first entity and a second entity means one of (1) the first entity, (2) the second entity, and (3) the first entity and the second entity. Multiple elements listed with “and/or” should be construed in the same fashion, i.e., “one or more” of the elements so conjoined.

As used herein, “exemplary” is used exclusively herein to mean “serving as an example, instance, or illustration.” Any aspect described herein as “exemplary” is not necessarily to be construed as preferred or advantageous over other aspects.

As used herein, “hydrocarbon” includes any of the following: oil (often referred to as petroleum), shale, oil sands, natural gas, gas condensate, tar, bitumen, and other known hydrocarbons.

As used herein, “hydrocarbon management” or “managing hydrocarbons” includes hydrocarbon extraction, hydrocarbon production, hydrocarbon exploration, identifying potential hydrocarbon resources, identifying well locations, determining well injection and/or extraction rates, identifying reservoir connectivity, acquiring, disposing of and/or abandoning hydrocarbon resources, reviewing prior hydrocarbon management decisions, and any other hydrocarbon-related acts or activities.

As used herein, “ecology” refers to the study of the interactions between the living and non-living components of a system. Ecology includes biology, microbiology and molecular biology. Ecology also includes parameters such as community composition, community structure, and function. Organism behavior and quantity, as well as metabolites and products are also important parameters. These parameters vary in response to how the biotic components interact with abiotic components. For example, various studies have shown that community composition and community structure can be strong indicators of past or ongoing chemical and physical processes and conditions.

As used herein, “community composition” refers to the organisms in the system (e.g., bacteria vs. archaea, species x vs. species y, etc.).

As used herein, “community structure” refers to the relative abundance of each type of organisms in the system (e.g., 10% bacteria and 90% archaea, 50% species x and 50% species y, etc.)

As used herein, “function” refers to both the state of an organism or community of organisms (e.g., dead vs. alive; active vs. inactive) and the metabolic processes occurring (e.g. hydrocarbon degradation, sulfate reduction, iron reduction, fermentation, etc.).

As used herein, “behavior” encompasses responses to stimuli such as motility, attachment (including biofilm formation), bioluminescence, mineral precipitation, spore formation, etc.

As used herein, “products” refer to proteins, lipids, exopolymeric substances, and other cellular components that organisms produce under a given set of conditions.

As used herein, “lipids” refers to hydrophobic or amphiphilic compounds that compose cell membranes of organisms, energy storage and signaling molecules.

As used herein, “in situ analysis” refers to the analysis of samples within the environment of interest. This approach is similar to other geochemical measurements, such as pH, temperature, pressure, concentration of dissolved ions, etc., which can be measured using a variety of in situ tools and probes.

As used herein, a “microarray” is a multiplex lab-on-a-chip that allows many tests to be performed simultaneously or in sequence. It is an array of hundreds to thousands of spots containing probes (or tags) of various types. Lab-on-a-chip and microfluidics devices allow for the analysis of samples using miniaturized laboratory processes, which require 10⁻⁶−<10⁻⁹ L samples.

As used herein, a “sensor” is a device that detects and measures different physical, chemical, and biological signals.

As used herein, “direct-sample probing” or “down-hole probing” refers to the characterization of a sample in its intact form, without extracting the important components. In both cases, a dye or other reactive material can be used to enhance the important characteristics.

As used herein, “ex situ analysis” refers to the analysis of samples outside of their original environment. Culture- or cell-based techniques require that live organisms be captured in order to further study them to make the appropriate assessments. Organisms are characterized for various phenotypes and physiological aspects. They are tested for their ability to survive and grow under a variety of environmental conditions such as pressure, temperature, salinity, etc. The ability of organisms to degrade hydrocarbons of interest is also determined. Organisms exhibiting target characteristics are also isolated and characterized at depth. Molecular characterization typically requires the extraction of components from samples. These components include nucleic acids (e.g., DNA and RNA), proteins, lipids, exopolymeric substances, etc. Analysis of these components requires various techniques which include nucleic acid sequencing, protein sequencing, and/or some sort of separation and/or hybridization.

As used herein, “sequencing” refers to the determination of the exact order of nucleotide bases in a strand of DNA (deoxyribonucleic acid) or RNA (ribonucleic acid) or the exact order of amino acids residues or peptides in a protein. Nucleic acid sequencing can be done using Sanger sequencing or next-generation high-throughput sequencing including but not limited to massively parallel pyrosequencing, Illumina sequencing, or SOLiD sequencing, ion semiconductor sequencing. Amino acid sequencing is done by mass spectrometry and Edman degradation.

As used herein, “genomics” refers to the study of genomes of organisms, which includes the determination of the entire DNA sequence of organisms as well as genetic mapping.

As used herein, “DNA analysis” refers to any technique used to amplify and/or sequence DNA within the samples. DNA amplification can be accomplished using PCR techniques or pyrosequencing. As a non-limiting example, sequencing the hyper-variable region of the 16S rDNA (ribosomal DNA) may be used for species identification via DNA.

As used herein, “RNA analysis” refers to any technique used to amplify and sequence RNA within the samples. The same techniques used to analyze DNA can be used to amplify and sequence RNA. RNA, which is less stable than DNA is the translation of DNA in response to a stimuli and thus is thought to provide a more accurate picture of the metabolically active members of the community.

As used herein, “transcriptomics” refers to the amplification and sequencing of mRNA (messenger RNA), rRNA (ribosomal RNA), and tRNA (transfer RNA). These types of RNA are used to build and synthesize proteins. Understanding what transcripts are being used allows us to understand what proteins are being produced. Transcriptomics provides information about the functional structure of an environment.

As used herein, “proteomics” refers to the description of proteins produced by bacteria and/or archaea. Proteins can be used to describe the function of the most active members of a microbial community. Proteomics can be used to describe community structure, but only if the links between individual species and expressed proteins are clearly understood. Proteins are separated using two dimensional electrophoresis. Then these proteins are analyzed using a TOF (time of flight) mass spectrometer coupled to a liquid chromatograph or a MALDI (matrix assisted laser desorption/ionization) unit. Since proteins cannot be easily amplified proteomic analysis in natural samples requires a lot of biomass to be successful.

As used herein, “lipid analysis” refers to quantification and description of the phospho-lipids present in a sample. Phospho-lipids are compounds containing two chains of hydrophobic compounds linked together by a hydrophilic head group. Different species of bacteria and archaea produce different types of lipids. Additionally, all known bacterial lipids are joined together with an ester bond while all known archaeal lipids are joined together with an ether bond. Intact lipids should provide information about current community structure. As lipid production may vary as a function of temperature, pressure and salinity, lipid analysis may provide information about reservoir conditions. While the hydrophilic head group in a lipid is easily degradable the remaining hydrophobic chains are quite stable. Derivatives of these chains are used as biomarkers in organic geochemistry to fingerprint oils. Unaltered lipids can be used in a similar matter. Altered lipids are often used to fingerprint oils in organic geochemistry. Non-intact lipids will provide information about community structure in the past. This will let us know if conditions were different at some point in the past. These compounds will allow us to identify areas of past microbiological activity where DNA based markers have already been destroyed.

As used herein, “metabolites” refer to compounds produced by bacteria and archaea during respiration or fermentation. Acetic acid is an example of a metabolite with commercial applications. Metabolites provide information about the type of hydrocarbon being used as a substrate as well as information about physical and chemical conditions in the reservoirs. Additionally, certain characteristics of community structure and function are likely to be indicative of hydrocarbon reservoirs. For example the presence of specific species and/or metabolites may indicate or infer the presence of hydrocarbons and/or conditions at depth. Detailed descriptions of sample ecology will highlight differences in indicator species, transcripts, lipids, proteins and metabolites that distinguish seeps connected to larger hydrocarbon reservoirs from seeps in which no reservoirs are present.

As used herein, “paleo-seep” refers to an area that is no longer seeping.

Physical and chemical conditions in hydrocarbon reservoirs are very different from conditions at the seafloor. Pressure and temperature are both generally higher in hydrocarbon reservoirs than at the seafloor. Additionally, salinity is often higher in hydrocarbon reservoirs and organic carbon is more abundant. Thermophilic and halophilic bacteria have been isolated from hydrocarbon reservoirs. If these organisms are transported to the surface they will be detectable in descriptions of ecology. Furthermore, organisms living at reservoir conditions and/or transported to the sea floor will express different proteins and lipids, thereby permitting a determination of reservoir pressure and temperature based on these variables. In the absence of an active hydrocarbon system, the links between the water column, seafloor sediment and subsurface ecology become less clear.

Furthermore, the relative contribution of reservoir ecology to the water column, seafloor sediment, and subsurface rock ecology can be linked to hydrocarbon migration pathways and therefore hydrocarbon system type can be inferred. Samples at seeps that are fed by hydrocarbon reservoirs will share some characteristics with samples taken directly from those reservoirs. The techniques described above may be combined with physical and chemical measurements to create a complete, coherent description of the ecology of a given sample. Samples that are physically connected will share ecological characteristics. For example, a sediment sample from a seep will share ecological characteristics with the reservoir where the seeping fluids originated. According to methodologies and techniques, a method is provided explaining how to describe the ecology of a sample and how to relate the ecology of physically disparate samples to determine the presence or absence of an active hydrocarbon system and physiochemical conditions associated with it.

Disclosed methodologies and techniques provide exploration information independent of currently used techniques. Alternatively, disclosed aspects supplement information currently collected to better improve decision making. Furthermore, disclosed aspects enable a more direct linkage of surface data to subsurface conditions.

According to aspects of disclosed methodologies, a method is provided for using the ecology of parts or all of a hydrocarbon system to determine characteristics of the system. Samples from various parts of the hydrocarbon system are measured, observed, and analyzed. Community structures and community functions are determined, and an ecology of the sample is derived. The sample ecology assists in determining the presence of a hydrocarbon reservoir, as well as characteristics of the reservoir.

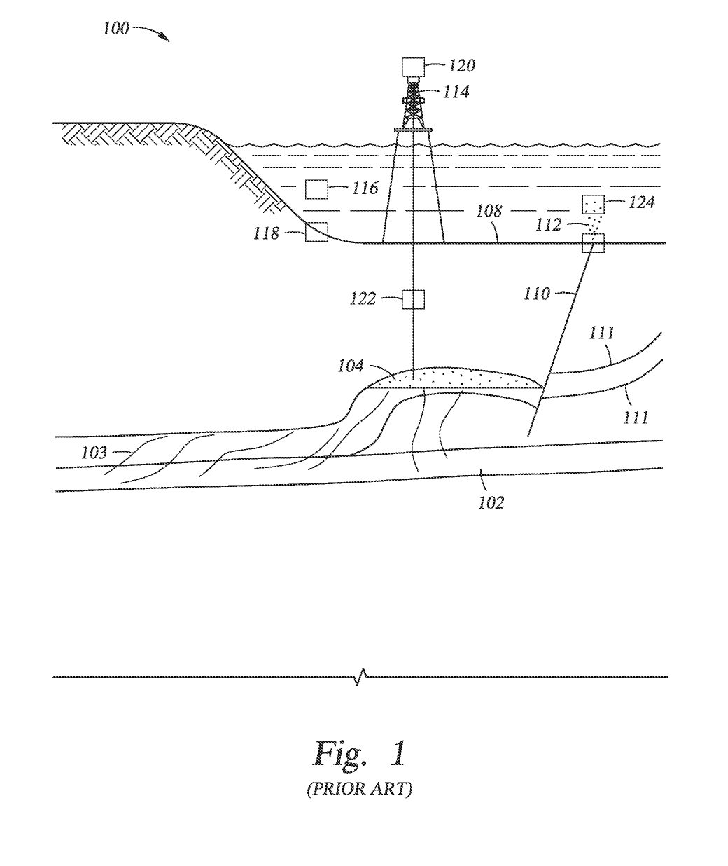

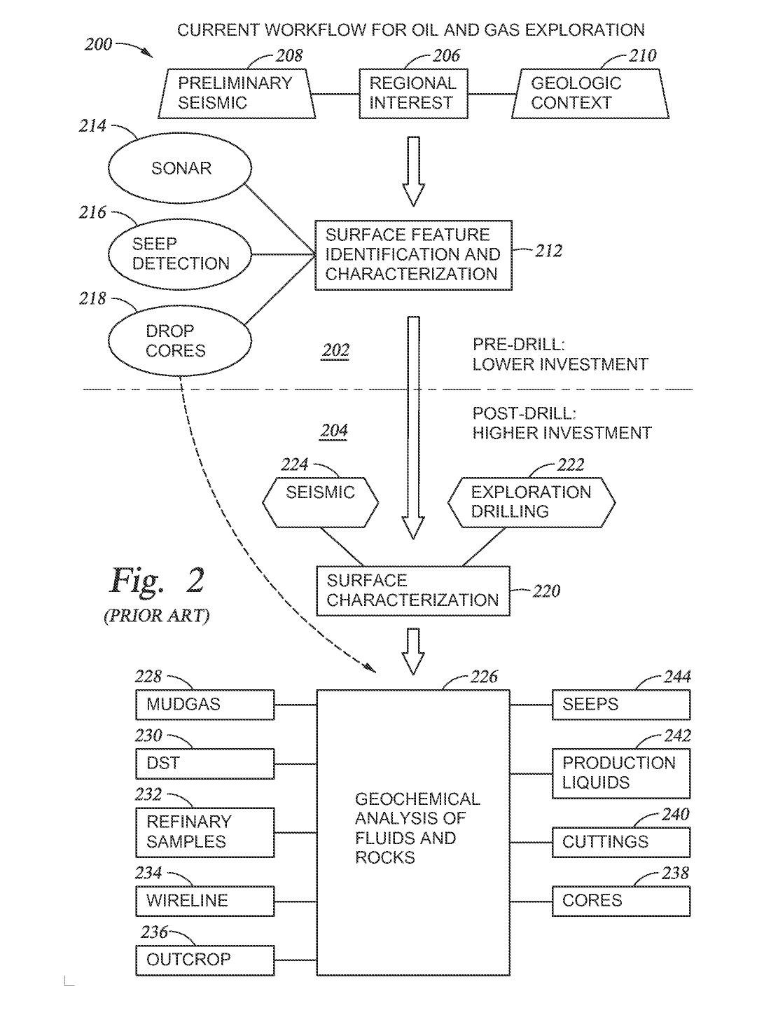

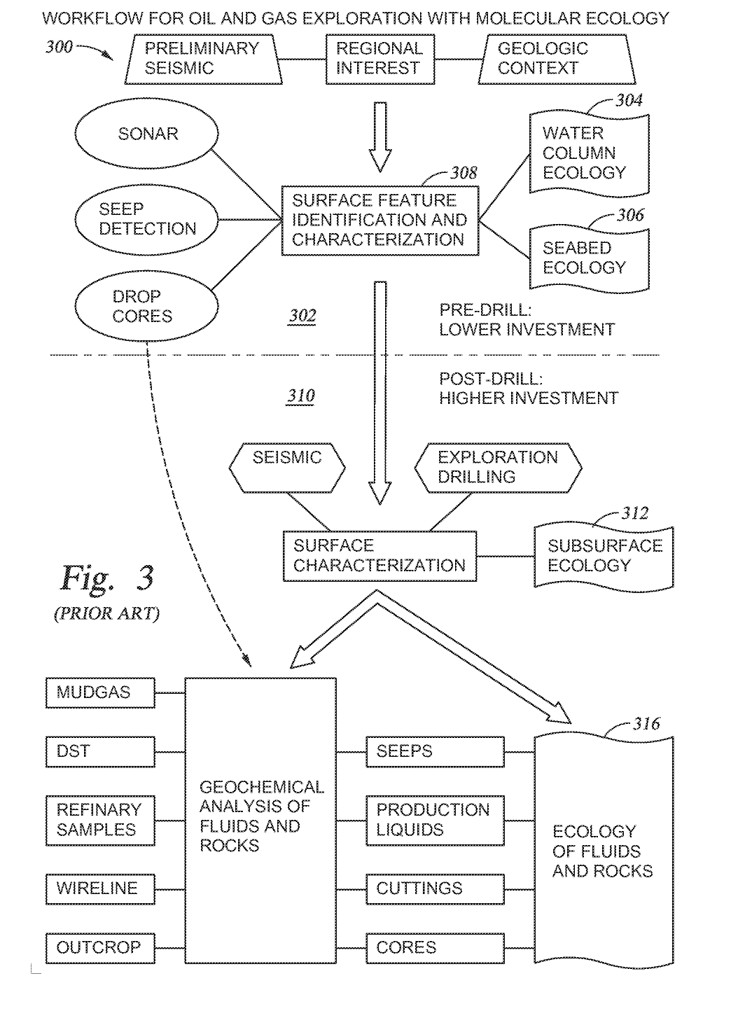

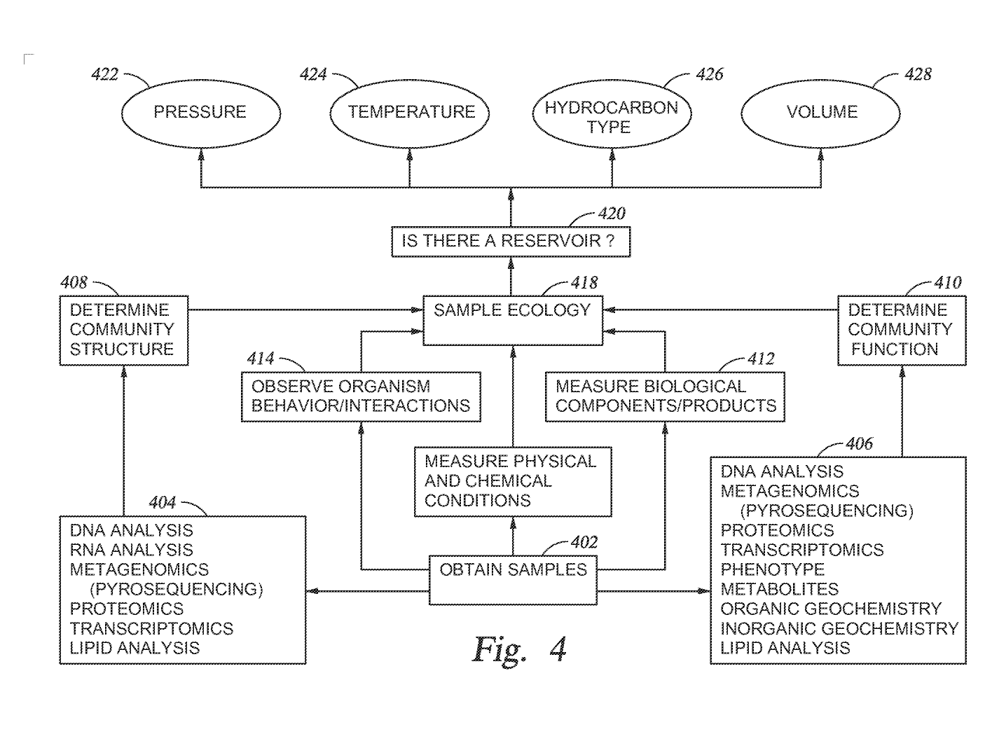

FIG. 4 is a schematic diagram of a method 400 according to disclosed methodologies and techniques. At block 402 samples are taken or collected from various aspects of a hydrocarbon system, such as system 100 in FIG. 1. As previously discussed, samples are collected from seafloor sediments (at 118) where there is evidence for active seepage. This evidence may include but is not limited to physical disturbance of the sediment, bubble trains, microbial mats, and oil slicks or sheens at the sea-air interface. Sediment samples are also collected from seafloor sediments (at 119) where there is no physical evidence of active seepage. Rock samples are collected from drill cores 122. Liquid samples are collected from the water column 114 above seeps 112 and/or from production platforms 120 where hydrocarbon reservoirs are actively being produced. Liquid samples may also be taken in areas where there is no physical evidence of active seepage, as shown at 116 in FIG. 1. Liquid samples may include water and hydrocarbon independently or in a mixture. To preserve ecology integrity where required, all sediment, water and rock samples are frozen as soon after collection as possible. The samples are maintained at a low temperature, which may be as low as −80° C., until analyses are performed. For samples not requiring freezing (in-situ analysis, for example), this step may be skipped.

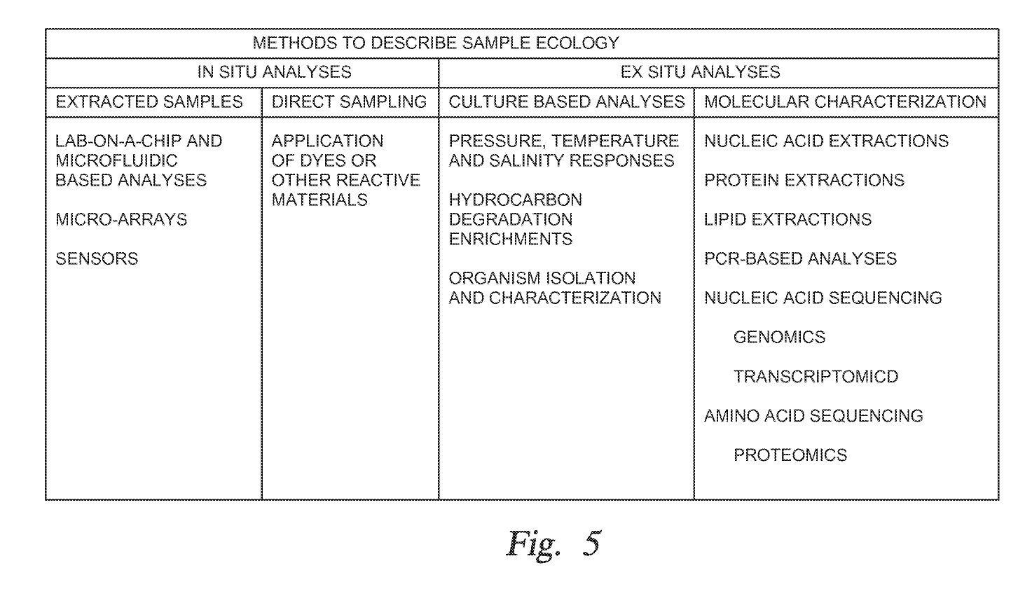

At blocks 404 and 406, the samples are analyzed using various methods to ascertain aspects of their ecology. The various methods may include DNA analysis, RNA analysis, metagenomics (including pyrosequencing), proteomics, transcriptomics, lipid analysis, phenotyping, metabolite analysis, organic geochemistry, and inorganic geochemistry. Other methods to describe sample ecology are shown in FIG. 5. Most of the methods shown in blocks 404 and 406 are classified as ex situ molecular characterization in FIG. 5. The methods in block 404 as well as any other methods in FIG. 5 are used to determine the community structure of the sample ecology, as indicated by block 408. The methods in block 406 as well as any other methods in FIG. 5 are used to determine the community function of the sample ecology, as indicated by block 410. As shown by block 412, measurement of biological components and/or processes may also assist in determining the community function of the sample ecology. For example, in the sediments and fluids surrounding a cold methane seep the following microorganisms might be found: Desulfobacterium anilini, Desulfovibrio gabonensis, Archaeoglobus fulgidus Methanobacterium ivanovii, ANME-1 (anaerobic methanotroph), and ANME-2. This list of species is the community structure (or composition). Genetic and culture-based information about these species informs us that segments of the community are reducing sulfate, oxidizing methane to CO₂, and reducing CO₂ back to methane. These metabolic activities describe the community function of this hypothetical community. Note that community structure does not necessarily imply knowledge about function. Likewise, geochemical or genetic information about function does not necessarily imply the presence or absence of specific species.

The determination of the community structure 408 and the community function 410 are used, together with observing organism behavior interaction (block 414), measured physical and chemical conditions (block 416), and measured biological components and products (block 412), to derive and understand the ecology of the samples (block 418). The sample ecology may then be used to determine whether the samples indicate the presence of a reservoir (block 420). Additionally, because the sample ecology may vary depending on pressure, temperature, hydrocarbon type, and volume, the sample ecology may assist in determining pressures (block 422), temperatures (block 424), hydrocarbon type (block 426), and volumes (block 428) associated with the sample and/or an associated reservoir.

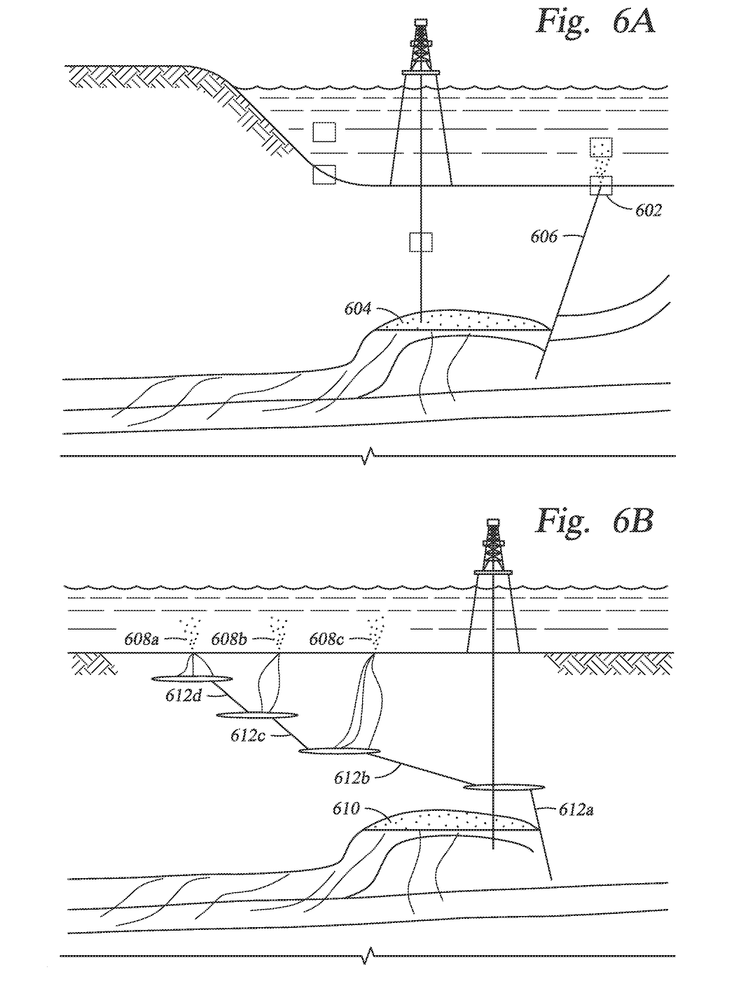

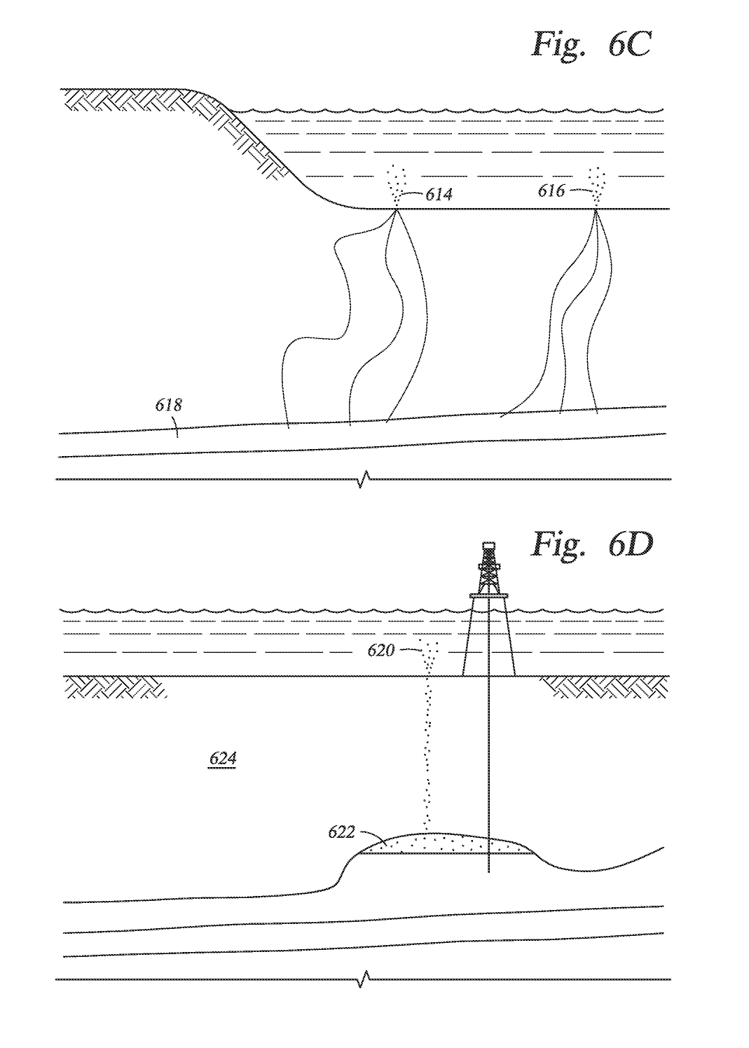

FIGS. 6A-6D show different types of seafloor hydrocarbon seeps. FIG. 6A is similar to FIG. 1, and shows a seep 602 directly connected to an economically viable hydrocarbon reservoir 604 through a fault 606. FIG. 6B shows a series of seeps 608a, 608b, 608c indirectly connected to an economically viable hydrocarbon reservoir 610 through a series of faults 612a, 612b, 612c, 612d. FIG. 6C shows a pair of seeps 614, 616, independent of any faults that are linked to an actively generating source rock 618 in which there is no reservoir.

FIG. 6D shows a fault-independent seep 620 associated with an economically viable hydrocarbon reservoir 622. The hydrocarbons in the reservoir overcome the capillary entry pressure of the overlying rock 624 and escape to the surface 626. Each of these seeps in FIGS. 6A-6D have different ecologies associated with the physical and chemical conditions unique to each system. Similarly, to the extent paleo-seeps and areas near seeps exhibit unique ecologies, the techniques described in this invention can be used to identify such paleo-seeps and/or areas near seeps.

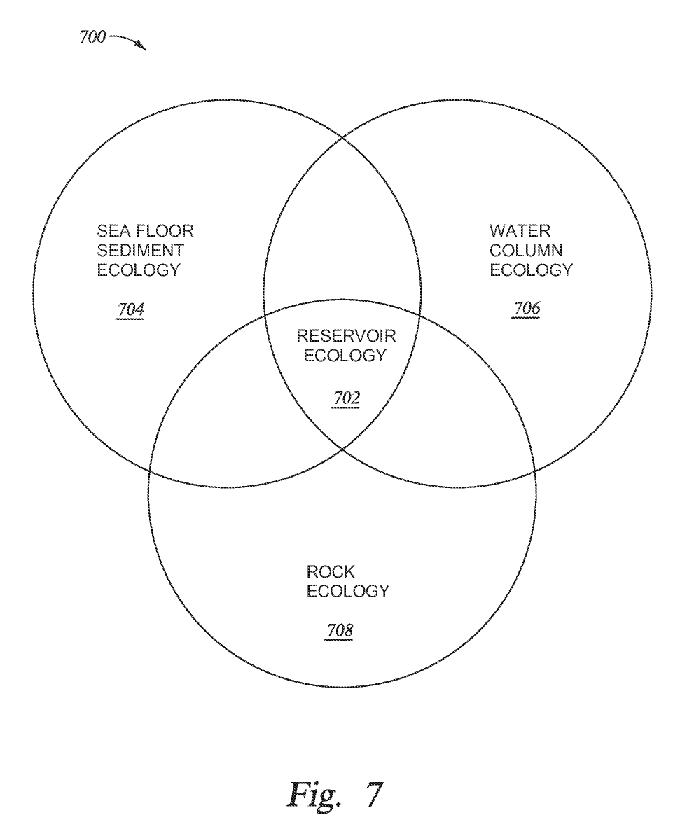

FIG. 7 is a diagram 700 demonstrating that reservoir ecology 702 can affect the ecology of associated environments like sea floor sediments surrounding a seep 704, the water column above a seep 706, and the ecology of rocks above the reservoir interval 708.

Throughout this disclosure the focus has been on the sampling and analysis of microbes. However, the disclosed methodologies and techniques can be applied to other organisms such as macro-algae, viruses, phages, fungi, chemosynthetic communities, and the like.

In an aspect of the disclosed methodologies and techniques, a fluid sample is collected from a reservoir with known physical and chemical conditions. The ecology of this sample is described using the techniques defined herein. A sediment sample is collected from a hydrocarbon seep connected to the known reservoir. The ecology of this seep is described in the same manner. Key species are identified via their DNA, RNA and lipids that link the two samples together. Additionally key community functions are identified via proteins, transcripts and metabolites that relate the two environments to each other. These links can be used in exploration settings where the links between seeps and reservoirs are less definitive.

The indicators developed herein can be used to identify seeps that are likely linked to reservoirs. Seeps that are fed from shallow deposits or directly from the source rock will not have the same set of characteristics. Additionally, ecology in seafloor sediments can be used to identify smaller seeps that do not have physical surface expressions.

Paleo-seeps can be identified via intact lipids and metabolites in sediments. These compounds are stable enough to remain in the sediment for years after active seeping has ceased. Lipid derived compounds are commonly used to fingerprint oils in organic geochemistry. These compounds are stable over geologic time scales. Paleo-seeps may be associated with economic hydrocarbon reservoirs that are no longer receiving new charge from the source rocks.

In addition to conventional exploration, the workflow and tool kit described herein can also provide critical information during unconventional exploration and development. Specifically, oil shale, shale gas and oil sand systems have properties that vary as a function of temperature, pressure, hydrocarbon type, inorganic mineralogy and chemistry. These properties can impact the predicted economic volumes that can be obtained from these unconventional reservoirs. Oil shale and shale gas are settings where the source rock is the reservoir, which means hydrocarbon migration is limited. Microbial products and biomarkers may help identify in situ pressures, temperatures and variations in hydrocarbon types across a geologic area of interest. Although this data would be obtained from test well samples, there is still an opportunity to calibrate basin and petroleum system models and constrain fluid or gas properties to better identify and extract resources. The role of indigenous microbial communities in controlling or altering the interface between mineral-hydrocarbon-aqueous phases may apply for the oil shale scenario, but are perhaps even more critical for oil sands. Typically, added microbial or fungal byproduct slurries are used to help alter subsurface conditions. This alteration is accomplished by the formation or addition of surfactants or by changing the hydrocarbon properties or composition. For example, converting viscous hydrocarbons to methane can help facilitate hydrocarbon extraction. The methodologies and techniques described herein may help optimize selection of zones, facies, or formations that have indigenous communities that may already produce or enhance hydrocarbon extraction without additional treatments. Specifically in oil sands, samples from multiple zones are combined to produce an aggregate that is then processed to remove the oil. If the proportions of these different materials are adjusted to include those that have increased natural surfactants, then this may increase the overall yield obtained from the homogenized aggregate.

If this data is tied to multiple other parameters that are indicative of pressure, temperature or salinity in the subsurface, then a more robust assessment may be made. Information about the reservoir, and the hydrocarbon system in general, may be missed by not incorporating or integrating other geological, geochemical and ecological information into traditional or currently existing workflows. This integrated approach is one option for which the workflow and toolkit described herein may be applied.

Although the disclosed methodologies and techniques may be applied advantageously to oil and gas exploration activities, there are other ways in which said methodologies and techniques may be used, such as microbially enhanced oil recovery due to production of methane via methanogenesis, exopolysaccharides and enzymes causing changes fluid properties (e.g., viscosity), addition of microbial slurries to enzyme-activated proppants, and surfactants that change the interface between the hydrocarbons and minerals (e.g., emulsion breakers), reducing waxy components and increasing flow. In some cases the need to obtain microbial information is related to the potential for scale formation, reservoir souring and pipeline corrosion if left untreated. Although reservoir fluid flow applications are based primarily on introducing biological tags downhole, critical information about how these biotechnology systems work may provide necessary insight into facies-specific properties and behavior, such as zones with unique indigenous ecology. From this type of data set, there is potential to target specific subsurface conditions or intervals and therefore optimize site selection based on a particular suite of desired properties. In all of these examples, a toolkit that appropriately identifies inherent and diagnostic information linked to ecologic and geochemical conditions in the subsurface will be helpful to de-risk some zones considered for exploring unconventional hydrocarbon plays or systems.

Previous studies have relied on one or two methods that are mostly tied to functional or genomic information, such as obtained from DNA or RNA, and possibly one stable isotopic (e.g., carbon) or molecular signature, such as lipids. The disclosed methodologies and techniques provide the first method that combines a full suite of geochemical and biological tools to identify organisms, their by-products, metabolites and the like that may be transported from the reservoir to the air-sediment or water-sediment interface with the fluids and hydrocarbons. This also includes differentiation of organisms living in association with the hydrocarbons, or related transported materials, at the interface that may shed light on hydrocarbon quality or changes therein due to transport and any degradation that may occur along the migration pathway. If extracellular DNA and other biomarkers are released within the reservoir, there is time for equilibration, reaction and association with reservoir geochemistry that may provide characteristic compositions that are retained during transport to surface and therefore provides more opportunity for assessing subsurface conditions. For example, the disclosed methodologies and techniques that combine metagenomic analysis, proteomics, lipid analysis, molecular geochemistry, biomarker and/or isotopic information will provide more information about the reservoir, ecology, and hydrocarbons and fluids therein than could be acquired from other approaches, such as PCR, quantitative PCR (qPCR), microarray or culturing methods alone.

Illustrative, non-exclusive examples of methods and products according to the present disclosure are presented in the following non-enumerated paragraphs. It is within the scope of the present disclosure that an individual step of a method recited herein, including in the following enumerated paragraphs, may additionally or alternatively be referred to as a “step for” performing the recited action.
A. A method of identifying a hydrocarbon system, comprising:
   obtaining a sample from an area of interest;
   using a first plurality of analyses to determine a community structure of an ecology of the sample;
   using a second plurality of analyses to determine a community function of the ecology of the sample;
   using the community structure and the community function to determine whether the ecology of the sample matches a characteristic ecology of a hydrocarbon system; and
   when the ecology of the sample matches the characteristic ecology, identifying the sample as part of the hydrocarbon system.
A1. The method as recited in paragraph A, wherein the sample is obtained from sediment near a hydrocarbon seep.
A2. The method as recited in any of paragraphs A-A1, wherein the hydrocarbon seep is a subsea seep.
A3. The method as recited in any of paragraphs A-A2, wherein the sample is obtained from sediment in area with no hydrocarbon seep.
A4. The method as recited in any of paragraphs A-A3, wherein the sample is obtained from sediment in an area near a paleo-seep.
A5. The method as recited in any of paragraphs A-A4, wherein the sample is obtained from a water column above a hydrocarbon seep.
A6. The method as recited in any of paragraphs A-A5, wherein the sample is obtained from a drill core sample.
A7. The method as recited in any of paragraphs A-A6, wherein the sample is obtained from produced reservoir fluids.
A8. The method as recited in any of paragraphs A-A7, wherein the sample is a first sample, and further comprising:
   obtaining second and third samples from two of sediment near a hydrocarbon seep, sediment in an area with no hydrocarbon seep, sediment near a paleo-seep, a water column above a hydrocarbon seep, a drill core sample, and produced reservoir fluids;
   using the first plurality of analyses to determine a community structure of an ecology of each of the samples;
   using the second plurality of analyses to determine a community function of the ecology of each of the samples;
   using the community structure and the community function to determine whether the ecology of each of the samples matches an anticipated characteristic of a hydrocarbon system; and
   when the ecology of each of the samples matches the anticipated characteristic, identifying the sample as part of the hydrocarbon system.
A9. The method as recited in any of paragraphs A-A8, further comprising preserving the obtained sample at a temperature at or lower than minus 60 degrees Celsius.
A10. The method as recited in any of paragraphs A-A9, wherein the temperature is at or lower than about −80 degrees Celsius.
A1. The method as recited in any of paragraphs A-A10, wherein the first plurality of analyses to determine the community structure of the ecology of the sample include one or more of
   DNA analysis,
   RNA analysis,
   metagenomics,
   proteomics,
   transcriptomics, and
   lipid analysis.
A12. The method as recited in any of paragraphs A-A11, wherein the second plurality of analyses to determine the community function of the ecology of the sample include three or more of
   DNA analysis,
   metagenomics,
   proteomics,
   transcriptomics,
   phenotypes,
   metabolites,
   organic geochemistry,
   inorganic geochemistry, and
   lipid analysis.
A13. The method as recited in any of paragraphs A-A12, further comprising using the ecology of the sample to determine an aspect of the hydrocarbon system.
A14. The method as recited in any of paragraphs A-A-13, wherein the aspect of the hydrocarbon system is one of pressure, temperature, salinity, reservoir volume, and hydrocarbon type.
A15. The method as recited in any of paragraphs A-A14, wherein the hydrocarbon system comprises a subsurface hydrocarbon reservoir with seepage to a seafloor via a fault or fracture zone.
A16. The method as recited in any of paragraphs A-A15, wherein the hydrocarbon system comprises a subsurface hydrocarbon reservoir with capillary seepage to a seafloor.
A17. The method as recited in any of paragraphs A-A16, wherein the hydrocarbon system comprises a region of source rock without a reservoir.
A18. The method as recited in any of paragraphs A-A17, wherein the hydrocarbon system comprises one of an oil shale deposit, a shale gas deposit, and an oil sands deposit.

The disclosed aspects, methodologies and techniques may be susceptible to various modifications, and alternative forms and have been shown only by way of example. The disclosed aspects, methodologies and techniques are not intended to be limited to the specifics of what is disclosed herein, but include all alternatives, modifications, and equivalents falling within the spirit and scope of the appended claims.

1. A method of identifying a hydrocarbon system, comprising:
obtaining a sample from an area of interest;
using a first plurality of analyses to determine a community structure of an ecology of the sample;
using a second plurality of analyses to determine a community function of the ecology of the sample;
using the community structure and the community function to determine whether the ecology of the sample matches a characteristic ecology of a hydrocarbon system; and
when the ecology of the sample matches the characteristic ecology, identifying the sample as part of the hydrocarbon system.

2. The method of claim 1, wherein the sample is obtained from sediment near a hydrocarbon seep.

3. The method of claim 2, wherein the hydrocarbon seep is a subsea seep.

4. The method of claim 1, wherein the sample is obtained from sediment in area with no hydrocarbon seep.

5. The method of claim 1, wherein the sample is obtained from sediment in an area near a paleo-seep.

6. The method of claim 1, wherein the sample is obtained from a water column above a hydrocarbon seep.

7. The method of claim 1, wherein the sample is obtained from a drill core sample.

8. The method of claim 1, wherein the sample is obtained from produced reservoir fluids.

9. The method of claim 1, wherein the sample is a first sample, and further comprising:
obtaining second and third samples from two of sediment near a hydrocarbon seep, sediment in an area with no hydrocarbon seep, sediment near a paleo-seep, a water column above a hydrocarbon seep, a drill core sample, and produced reservoir fluids;
using the first plurality of analyses to determine a community structure of an ecology of each of the samples;
using the second plurality of analyses to determine a community function of the ecology of each of the samples;
using the community structure and the community function to determine whether the ecology of each of the samples matches an anticipated characteristic of a hydrocarbon system; and
when the ecology of each of the samples matches the anticipated characteristic, identifying the sample as part of the hydrocarbon system.

10. The method of claim 1, further comprising preserving the obtained sample at a temperature at or lower than minus 60 degrees Celsius.

11. The method of claim 10, wherein the temperature is at or lower than about −80 degrees Celsius.

12. The method of claim 1, wherein the first plurality of analyses to determine the community structure of the ecology of the sample include one or more of
DNA analysis,
RNA analysis,
metagenomics,
proteomics,
transcriptomics, and
lipid analysis.

13. The method of claim 1, wherein the second plurality of analyses to determine the community function of the ecology of the sample include three or more of
DNA analysis,
metagenomics,
proteomics,
transcriptomics,
phenotypes,
metabolites,
organic geochemistry,
inorganic geochemistry, and
lipid analysis.

14. The method of claim 1, further comprising using the ecology of the sample to determine an aspect of the hydrocarbon system.

15. The method of claim 14, wherein the aspect of the hydrocarbon system is one of pressure, temperature, salinity, reservoir volume, and hydrocarbon type.

16. The method of claim 1, wherein the hydrocarbon system comprises a subsurface hydrocarbon reservoir with seepage to a seafloor via a fault or fracture zone.

17. The method of claim 1, wherein the hydrocarbon system comprises a subsurface hydrocarbon reservoir with capillary seepage to a seafloor.

18. The method of claim 1, wherein the hydrocarbon system comprises a region of source rock without a reservoir.

19. The method of claim 1, wherein the hydrocarbon system comprises one of an oil shale deposit, a shale gas deposit, and an oil sands deposit.
